(19) United States
(12) Patent Application Publication
(10) Pub. No.: US 2006/0074477 A1
(43) Pub. Date: Apr. 6, 2006

(54) Self-expanding stent delivery system

(75) Inventors: William A. Berthiaume, Santa Rosa, CA (US); Christopher T. Collins, San Rafael, CA (US); Don Tran, Novato, CA (US); Martha Barajas-Torres, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/954,123

(22) Filed: Sep. 29, 2004

(51) Int. Cl. A61F 2/06

(52) U.S. Cl. 623/1.11

(57) ABSTRACT

A catheter for a self-expanding stent includes a proximal shaft with a longitudinal guide way along the length of the proximal shaft, a guide member for accessing a guidewire lumen defined by the proximal shaft and for drawing a guidewire into and out of the guidewire lumen along the proximal shaft. In one aspect, the catheter includes a proximal sliding mechanism coupled to a link, which is coupled to a distal outer shaft retaining a self-expanding stent in a compressed state. The sliding mechanism draws the link proximally, which draws the distal outer shaft proximally thereby allowing the stent to radially expand. In another aspect, the proximal sliding mechanism is coupled to a proximal shaft, which is coupled to the distal outer shaft. The sliding mechanism draws the proximal shaft proximally, which draws the distal outer shaft proximally to thereby allow the stent to radial expand.

### FIELD OF THE INVENTION

The present invention relates to a medical device. More specifically, the invention relates to a catheter, which includes a multi-exchange (MX) component and stent delivery system.

### BACKGROUND OF THE INVENTION

Cardiovascular disease, including atherosclerosis, is a leading cause of death in the U.S. One method for treating atherosclerosis and other forms of coronary narrowing is percutaneous transluminal coronary angioplasty, commonly referred to as “angioplasty” or “PTCA”. The objective in angioplasty is to enlarge the affected coronary vessel from within by radial expansion. The procedure is accomplished by inflating a balloon of a balloon catheter within the narrowed lumen. The balloon expands in the coronary vessel in several different radial dimensions and opens the vessel based on the nature of the plaque. For example, soft, fatty plaque deposits are flattened by the balloon, while hardened deposits are cracked and split to enlarge the lumen.

More than one procedure may be necessary to effectively dilate the coronary vessel. For example, often successive dilatations using balloon catheters with balloons of increasingly larger diameters may be required. Once dilation is complete, to help prevent closure of the vessel, reinforce a vessel wall, and/or prevent restenosis, an intravascular prosthesis, or a stent, is generally implanted inside the lumen. The stent may be a self-expanding stent or a balloon-expandable stent. Once the stent is in place, the procedure may be repeated in other narrowed lumen areas of the coronary vessel. Stents may also be used to open narrowed coronary vessels without first using a balloon catheter to open the lumen, for example when damage rather than plaque is the cause of the narrowed vessel.

Generally, a guidewire is first fed through the vasculature to the treatment site and the catheter is tracked along the guidewire in to the correct position within the vessel. In order to accomplish multiple dilatations and/or one or more stent placements, the original catheter must be removed, and additional catheters must be tracked to the treatment site. When catheters are exchanged, it is advantageous to leave the guidewire in place while the first catheter is removed and to insert the second catheter over the same guidewire. Thus, there is no need to reestablish the path to the treatment site through the vasculature by inserting a new guidewire. To remove a catheter while leaving the guidewire in place, however, a clinician must maintain control of the proximal end of the guidewire while the catheter is retracted proximally and removed.

There are several types of catheters commonly used in angioplasty procedures. An over-the-wire (OTW) catheter includes a guidewire lumen that runs the entire length of the catheter and is attached to, or enveloped within, a catheter shaft. Thus, the entire length of an OTW catheter is tracked over a guidewire.

If a catheter exchange is required while using a standard OTW catheter, the clinician must add an extension to the proximal end of the guidewire or have a very long guidewire in order to maintain control of the proximal end of the guidewire as the entire OTW catheter is retracted and removed. A second or subsequent catheter is then backloaded onto the proximal end of the guidewire and tracked to the treatment site. Multiple operators are required to hold the extended or very long guidewire in place and maintain its sterility while catheters are exchanged.

A rapid-exchange (RX) catheter avoids the need for multiple operators when exchanging the catheter and therefore is often referred to as a “single operator” catheter. With a RX catheter, the guidewire runs along the outside of the catheter, except where it enters a short guidewire lumen that extends within only a comparatively short segment of the catheter at a distalmost end of the catheter shaft. The guidewire can be held in place without an extension when the RX catheter is removed from the body. Once the original RX catheter is removed, a subsequent RX catheter may be threaded onto the in-dwelling guidewire and tracked to the treatment site. Although the RX catheter may provide the advantages discussed above, it presents several difficulties.

Without a full-length guidewire lumen, the proximal shaft of a RX catheter lacks an OTW catheter's stiffness and optimal transmission of force along the catheter length, which aids a clinician when pushing the distal end of the catheter through tightly narrowed and/or tortuous blood vessels. Even if the catheter buckles slightly when the distal tip of an OTW catheter is forced through a narrowed area, there is very little misalignment with the guidewire, such that most of the push force is transmitted to the distal tip.

Further, it is not possible to exchange guidewires with a RX catheter. A guidewire exchange may be needed when, for example, the guidewire becomes damaged during the procedure; a different shape, length, or size of guidewire is needed; and/or the guidewire is unintentionally withdrawn. A guidewire cannot be directed back into the RX catheter's proximal guidewire port, which is located at a distal end of the RX catheter positioned near the treatment site within the patient.

At the proximal end, the RX catheter and the guide wire extend side-by-side from the body of a patient, making it awkward to seal the system against blood loss during manipulation of the components. The sealing, or “anti-backbleed” function is typically accomplished with a “Tuohy-Borst” fitting that has a manually adjustable gasket with a hole that does not conform well to the side-by-side arrangement of a catheter shaft and guide wire.

Further, the lack of a full-length guide wire lumen in a RX catheter deprives the clinician of an additional lumen that may be used for other purposes, such as pressure measurement, injection of contrast dye distal to the stenosis, or infusing a drug.

A catheter capable of faster and more simple guidewire and catheter exchange than an OTW catheter and that addresses the deficiencies of a RX catheter is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”) and is disclosed in U.S. Pat. No. 4,988,356 to Crittenden et al., U.S. Application Publication Nos: 2003/0191491 to Duane; 2004/0059291 to MacDonnell et al.; 2004/0122363 to Gribbons et al.; and U.S. patent application Ser. No. 10/722,191 filed Nov. 24, 2003. The above noted patent and applications are each incorporated by reference in their entirety herein.

The MX catheter includes a catheter shaft having a guide way that extends longitudinally along the catheter shaft and that extends radially from a guidewire lumen to an outer surface of the catheter shaft. A guide member through which the shaft is slidably coupled cooperates with the longitudinal guide way such that a guidewire may extend transversely into or out of the guidewire lumen at any location along the longitudinal guide way's length. By moving the shaft with respect to the guide member, the effective over-the-wire length of the MX catheter is adjustable.

When using the MX catheter, the guidewire is maneuvered through the patient's vascular system such that a distal end of the guidewire is positioned across the treatment site. With the guide member positioned near a distal end of the catheter, a proximal end of the guidewire is threaded into a guidewire lumen opening at the distal end of the catheter and out through a proximal end of the guide member. By securing the guide member and the proximal end of the guidewire in a fixed position, the catheter may then be tracked over the guidewire by advancing the catheter first through the guide member and into the vasculature. In doing so, as the catheter advances, the guide member opens the guidewire lumen, feeds the guidewire into the lumen and closes the lumen again so that the advancing guidewire lumen envelops the guidewire. The MX catheter may be advanced over the guidewire in this manner until the distal end of the catheter is positioned at the treatment site and nearly the entire length of the guidewire is enveloped within the guidewire lumen.

Furthermore, the MX catheter may be exchanged with another catheter by reversing the operation described above and withdrawing the proximal end of the catheter from the patient while holding the proximal end of the guidewire and the guide member in a fixed position. As the MX catheter is retracted, the guide member opens the guidewire lumen, leads the guidewire from the lumen, and closes the lumen. When the catheter has been withdrawn only the distal portion of the catheter is still over the guidewire. The distal portion is of a sufficiently short length that the MX catheter may be drawn over the proximal end of the guidewire without releasing control of the guidewire or disturbing its position within the patient. The MX catheter permits catheter exchange without the very long or extended guidewire of the OTW catheter, without requiring withdrawal of the initially placed guidewire, and without many of the other difficulties discussed in association with RX catheters.

Heretofore, self-expanding stent delivery systems have incorporated either OTW or RX catheter systems, and clinicians have experienced the difficulties described above with respect to both OTW and RX catheters. Therefore, there exists the need to provide a self-expanding stent delivery system including the herein described benefits of a multi-exchange catheter.

### SUMMARY OF THE INVENTION

The catheter of the present invention provides a catheter that allows for easy guidewire exchange, easy catheter exchange, and accurate self-expanding stent deployment, by introducing a catheter for a self-expanding stent that features an MX catheter design. The catheter of the present invention includes a proximal shaft including a longitudinal guide way along the length of the proximal shaft. The catheter also includes a guide member for accessing a guidewire lumen defined by the proximal shaft and for drawing a guidewire into and out of the guidewire lumen as the guide member moves along the proximal shaft.

In one aspect of the present invention, the catheter includes a sliding mechanism positioned near a proximal end of the catheter. The sliding mechanism is coupled to a proximal end of a link, which has a distal end coupled to a distal outer shaft located near a distal end of the catheter. When a stent is properly positioned over a treatment site, the sliding mechanism draws the link proximally, which draws the distal outer shaft proximally. Since the distal outer shaft retains a self-expanding stent in a compressed state when it is in a distal position, the stent radially expands once the distal outer shaft has been retracted proximally to a proximal position.

In another aspect of the present invention, the sliding mechanism is coupled to a proximal end of the proximal shaft, which is coupled at a distal end to the distal outer shaft. In this case, the sliding mechanism draws the proximal shaft proximally, which draws the distal outer shaft proximally. As the distal outer shaft is retracted to a proximal position, the self-expanding stent is allowed to radially expand.

Other aspects of the present invention include, but are not limited to, using different types of guide members with a catheter of the present invention, a stop to keep the stent from moving proximally as the distal outer shaft is retracted, maintaining a distal inner shaft in a distal position, such that the entire catheter is not retracted when distal outer shaft is retracted, radiopaque markers, and a valve relief to minimize backbleed as the catheter is moved into and out of the vasculature.

### BRIEF DESCRIPTION OF THE FIGURES

The foregoing and other features and advantages of the invention will be apparent from the following, more particular description of embodiments of the invention, as illustrated in the accompanying drawings.

FIG. 1 is a perspective view of a catheter according to the present invention.

FIG. 2 is a sectional view along line A-A in FIG. 1 of a portion of the catheter in FIG. 1.

FIG. 3 is a sectional view along line B-B in FIG. 1 of a portion of the catheter in FIG. 1.

FIG. 4A is a cross-sectional view along line C-C in FIG. 3 of the catheter in FIG. 1.

FIG. 4B is a cross-sectional view along line D-D in FIG. 3 of the catheter in FIG. 1.

FIG. 5 is a perspective view of an embodiment of a guide member of the present invention.

FIG. 6 is a sectional view taken along line E-E of the guide member of FIG. 5.

FIG. 7 is a cross-sectional view taken along line F-F of the guide member of FIG. 5.

FIG. 8 is a perspective view of an alternative embodiment of a guide member of the present invention.

FIG. 9 is a perspective view of a main body portion of the guide member of FIG. 8.

FIG. 10 is a perspective view of an inner body portion of the guide member of FIG. 8.

FIG. 11 is a sectional view taken along line G-G of the inner body portion of FIG. 10.

FIG. 12 is a perspective view of an alternative embodiment of a guide member of the present invention.

FIG. 13 is a sectional view taken along line H-H of the guide member of FIG. 12.

FIG. 14 is a perspective view of an alternative embodiment of a guide member of the present invention.

FIG. 15 is a sectional view taken along line I-I of the guide member of FIG. 14.

FIG. 16 is cross-sectional view taken along line J-J of the guide member of FIG. 14.

FIG. 17 is a side partial plan and partial cross-sectional view of an alterative embodiment of a catheter of the present invention.

FIG. 18 is a sectional view of a distal portion of the catheter of FIG. 17.

FIG. 18A is a cross-sectional view taken along line K-K of the distal portion of FIG. 17.

FIG. 18B is a cross-sectional view taken along line L-L of the distal portion of FIG. 17.

FIG. 19 is a sectional view of a bond area of the distal portion of FIG. 17.

FIG. 20 is a sectional view of a bond region of the distal portion of FIG. 18.

FIG. 21 is a sectional view of a proximal portion of the catheter of FIG. 17.

FIG. 22 is a sectional view of the sliding mechanism of FIG. 21.

FIG. 23 is a sectional view of a bonding region of the sliding mechanism of FIG. 22.

### DETAILED DESCRIPTION OF THE INVENTION

The present invention is now described with reference to the figures where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

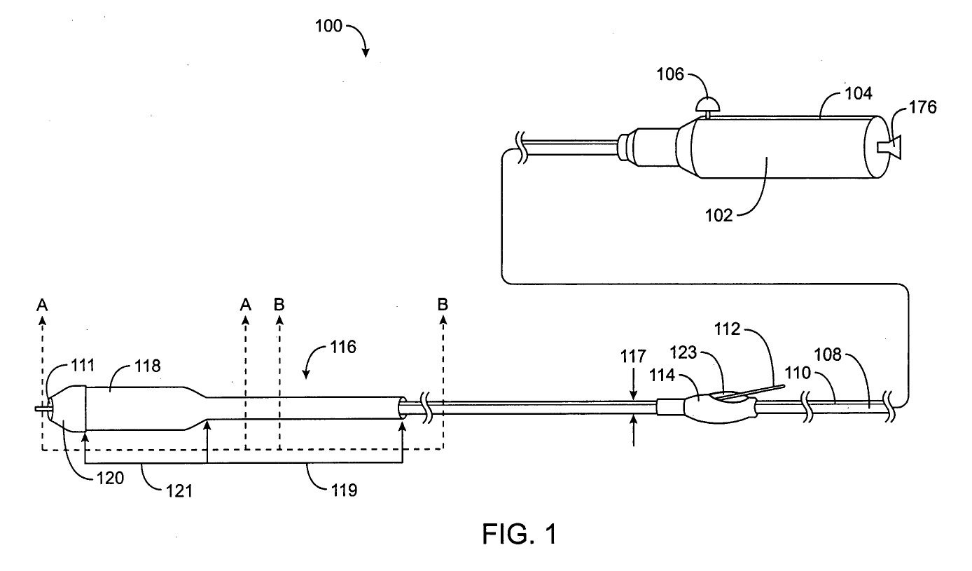

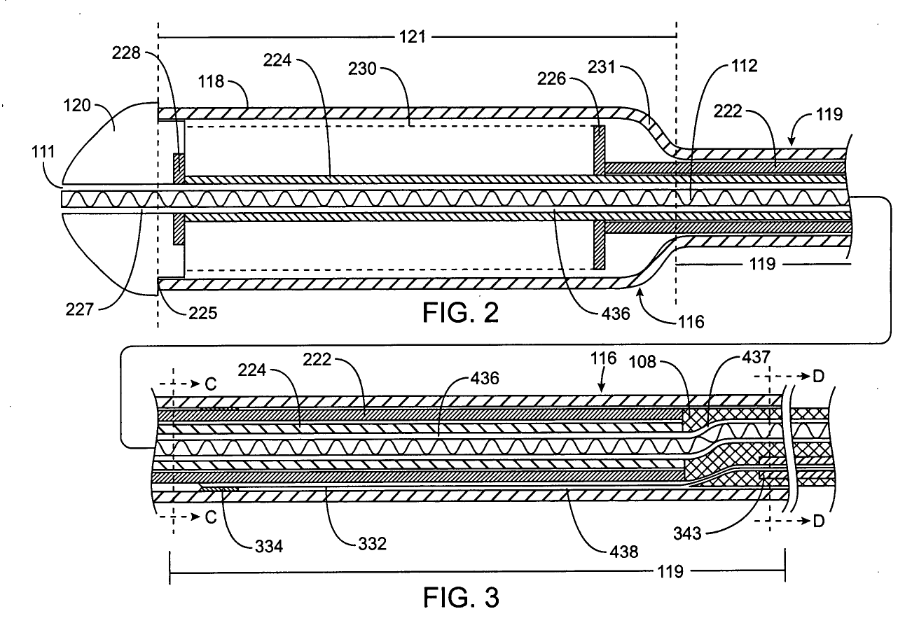

FIGS. 1-3 generally illustrate an embodiment of a self-expanding stent delivery catheter 100 according to the present invention. Catheter 100 includes at least a handle 102, a proximal shaft 108, a distal outer shaft 116 coaxial to and capable of sliding over proximal shaft 108, a link 332 between handle 102 and distal outer shaft 116, and a self-expanding stent 230 retained in a collapsed state by distal outer shaft 116.

Handle 102 includes a longitudinal opening 104 through which a sliding mechanism 106 slides proximally (towards the clinician) and distally (towards the treatment site). Sliding mechanism 106 is coupled to a proximal end of link 332. A distal end of link 332 is coupled to an anchor joint 334 of distal outer shaft 116.

Catheter 100 is advanced through the vasculature with distal outer shaft 116 in a distal position. When catheter 100 reaches the treatment site an operator slides sliding mechanism 106 proximally. As sliding mechanism 106 longitudinally slides, link 332 is pulled in a proximal direction, thereby sliding distal outer shaft 116 in a proximal direction. Since distal outer shaft 116 holds self-expanding stent 230 in a collapsed state, retracting distal outer shaft 116 mechanically deploys self-expanding stent 230. Handle 102 and sliding mechanism 106 may be made from a thermoformed or injection-molded thermoplastic material, for example, acrylonitrile-butadiene-styrene (ABS), a high density polyethylene or polycarbonate.

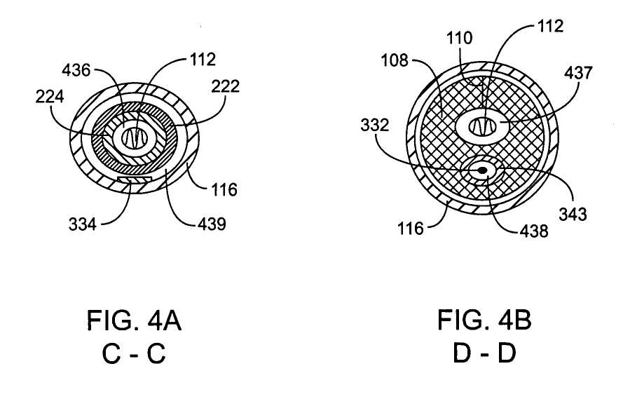

The distal end of handle 102 is coupled to a proximal end of proximal shaft 108 via a heat or ultrasonic weld or an adhesive treatment as would be known to one of ordinary skill in the art. With reference to FIGS. 4A and 4B, proximal shaft 108 defines a guidewire lumen 437 extending along the entire length of proximal shaft 108 for enclosing a guidewire 112. A longitudinal guide way 110 extends from substantially the proximal end to the distal end of proximal shaft 108 and radially from guidewire lumen 437 to an outer surface of proximal shaft 108. Guidewire lumen 437 is positioned adjacent to a link lumen 438 along proximal shaft 108. As shown in FIG. 4B, link lumen 438 also extends longitudinally along proximal shaft 108 from the proximal end to the distal end thereof. Link lumen 438 may also be defined by a support shaft 343 embedded within proximal shaft 108 to provide stiffness and strength when advancing proximal shaft through the vasculature.

Proximal shaft 108 may be, for example, an extruded, molded or thermoformed polymer, such as a nylon, high density polyethylene or PEBAX. Longitudinal guideway 110 may be formed, for example, using either a single, moveable blade, a “collet”-type arrangement of numerous blades, or by directional laser. Proximal shaft guidewire lumen 437 and link lumen 438 may have inside diameters of, for example, from about 0.015 inches to about 0.025 inches. Proximal shaft 108 may be, for example, oval in shape, with an outside diameter of about 0.030 inches to about 0.040 inches by about 0.055 inches to about 0.06 inches.

The distal end of proximal shaft 108 is attached to a proximal end of a distal inner shaft 224 and a spacer shaft 222. The shafts are bonded together using a heat or ultrasonic weld or an adhesive treatment as would be known to one of ordinary skill in the art. Support mandrels and/or wires may be placed within each shaft during bonding to ensure that the guidewire lumen 437 and link lumen 438 remain open during the bonding process and to provide a smooth transition between the lumens of proximal shaft and distal inner shaft 224.

Distal inner shaft 224 also includes a guidewire lumen 436. Guidewire lumen 436 runs longitudinally through distal inner shaft 224 and communicates with guidewire lumen 437 of proximal shaft 108. Distal inner shaft 224 may be manufactured, for example, by extruding, molding or thermoforming a polymer, such as nylon, high density polyethylene, or PEBAX. In another embodiment, distal inner shaft 224 includes a reinforcing component (not shown), such as a wire braid. Distal inner shaft 224 may have an inside diameter of, for example, about 0.015 inches to about 0.025 inches and an outside diameter of, for example, about 0.025 inches to about 0.030 inches.

A distal end of distal inner shaft 224 is coupled to a proximal end of a catheter tip 120. Catheter tip 120 also includes a guidewire lumen 227 extending therethrough and communicating with guidewire lumen 436 of distal inner shaft 224. Catheter tip 120 has a tapered distal end that includes a guidewire exit port 111, where catheter 100 is fed over guidewire 112. Catheter tip 120 may be a thermoplastic material, for example, nylon, high density polyethylene or PEBAX, or an elastomer, and may be bonded with the distal end of distal inner shaft 224 using a heat or ultrasonic weld or an adhesive treatment as would be known to one of ordinary skill in the art. Catheter tip 120 also includes a radiopaque marker 228, for example, at a distal or proximal end of catheter tip 120. Catheter tip 120 also includes a groove 225. Groove 225 shoulders the distal end of distal outer shaft 116, when in a distal position.

Distal outer shaft 116 is longitudinally slidable along an exterior of proximal shaft 108, spacer shaft 222 and distal inner shaft 224 and includes a proximal portion 119 and a bump distal portion 121. As shown in FIG. 3, proximal portion 119 of distal outer shaft 116 is coaxial with spacer shaft 222, distal inner shaft 224, and a portion of proximal shaft 108. Proximal portion 119 of distal outer shaft 116 has an inner diameter that is slightly larger than the outer diameter of spacer shaft 222 and proximal shaft 108. The annular space 439 created between the inner surface of distal outer shaft 116 and the outer surface of spacer shaft 222 and proximal shaft 108 respectively allows distal outer shaft 116 to slide longitudinally relative to both spacer shaft 222 and proximal shaft 108. This annular space 439 also communicates with link lumen 438 of proximal shaft 108 such that link 332 runs from link lumen 438 into this annular space, where it is coupled to distal outer shaft 116. Link 332 may be any suitable material of implant quality.

Located on the inner surface of proximal portion 119 of distal outer shaft 116 is anchor joint 334. Anchor joint 334 secures the distal end of link 332 to distal outer shaft 116. Anchor joint 334 is manufactured by trapping, laminating or bonding link 332 against the inner surface of distal outer shaft 116. Link 332 may be a pullwire as shown in FIG. 3. Alternatively, link 332 may be a pulltube, for example, a flush hypotube, or another mechanical connection. A pulltube serves the dual purpose of retracting distal outer shaft 116 and providing a manner for administering fluids, such as saline, dyes or drugs to the treatment site, for example, from a luer 176 at a proximal end of handle 102.

Bump distal portion 121 of distal outer shaft 116 includes a transition area 231 and a sheath portion 118. The distal end of proximal portion 119 of distal outer shaft 116 is connected to transition area 231 at a proximal end of bump distal portion 121. At transition area 231 an outer diameter of proximal portion 119 bumps radially, or increases, as the transition area 231 extends in the distal direction. As such, sheath portion 118 has a larger diameter where transitional area 231 ends, since sheath portion 118 contains self-expanding stent 230. The distal end of sheath portion 118 slides into and is secured in groove 225 of catheter tip 120. An inner surface of sheath portion 118 is coated with a lubricious solution, for example, silicone or a hydrophilic material, to allow the coated inner surface to easily slide relative to self-expanding stent 230.

Distal outer shaft 116 may be extruded, molded or thermoformed from a polymer, for example, nylon, high density polyethylene or PEBAX. In one embodiment, distal outer shaft 116 includes a reinforcing component (not shown), such as a wire braid. Proximal portion 119 may have an outside diameter of, for example, about 0.035 inches to about 0.06 inches, and sheath portion 118 may have an outside diameter of, for example, about 0.065 inches to about 0.080 inches.

Spacer shaft 222 is coupled to distal inner shaft 224 around an exterior of distal inner shaft 224. As illustrated in FIG. 3, a proximal end of proximal radiopaque marker 226 is coupled to or embedded within the distal end of spacer shaft 222. The distal end of proximal radiopaque marker 226 acts as a stop to prevent proximal movement of stent 230. Alternatively, spacer shaft 222 may include a stop flange (not shown). Proximal radiopaque marker 226 may therefore be coupled to or embedded into the stop flange. The stop flange alternatively prevents proximal movement of stent 230.

Stent 230 is positioned around distal inner shaft 224 and longitudinally between the distal end of spacer shaft 222 and the proximal end of catheter tip 120. The outer surface of stent 230 contacts the inner surface of distal outer shaft 116 along sheath portion 118.

Stent 230 is any suitable self-expanding stent manufactured from implant quality material. During catheter manufacture, stent 230 may be inserted into a distal end of sheath portion 118 between sheath portion of 118 of distal outer shaft 116 and distal inner shaft 224. In doing so, stent 230 is compressed and rolled down to a small profile. Catheter tip 120 is then secured to the catheter to close the distal end thereof.

Slidably attached to proximal shaft 108 is guide member 114. Guide member 114 has a guidewire exit lumen 123, which allows guidewire 112 to pass therethrough. Additionally, guide member 114 has an inner diameter 517, which may be, for example, oval-shaped if proximal shaft 108 is oval-shaped, and which is slightly greater than an outer diameter 117 of proximal shaft 108, so that guide member 114 may slide with respect to proximal shaft 108. Guide member 114 cooperates with longitudinal guide way 110 in proximal shaft 108 such that guidewire 112 may traverse into or out of guidewire lumen 437 of proximal shaft 108 at any location along the length of longitudinal guide way 110.

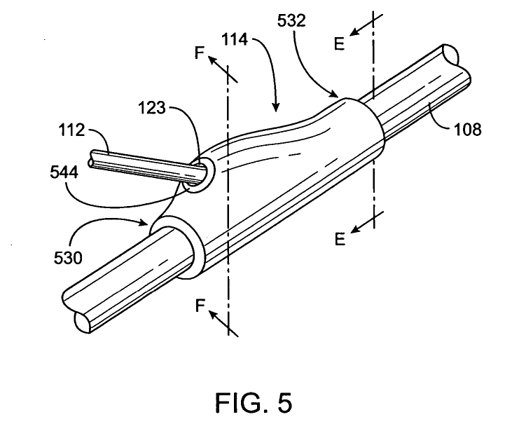

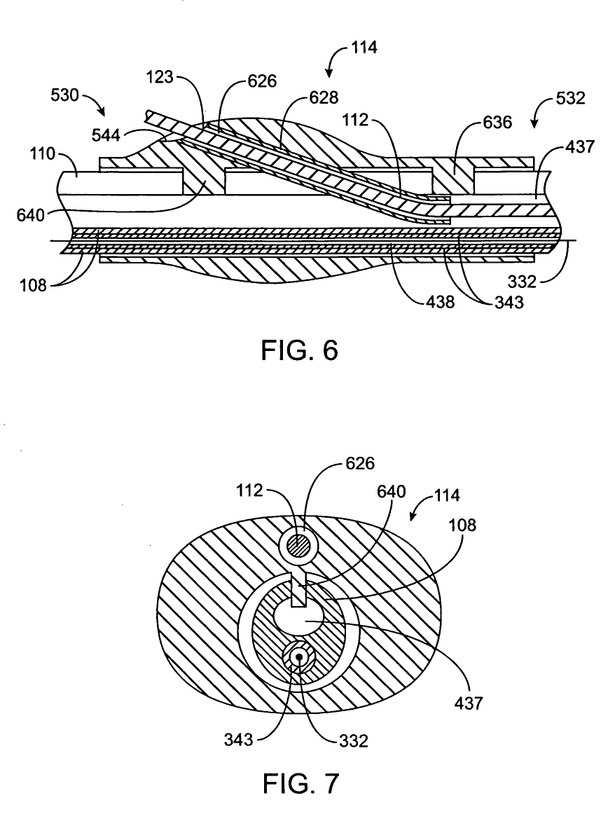

FIGS. 5-7 depict guide member 114. FIG. 6 is a sectional view taken along line E-E in FIG. 5, and FIG. 7 is a cross-sectional view taken along a line F-F in FIG. 5. Guide member 114 includes guidewire exit port 123 directing guidewire 112 to enter and exit proximal shaft 108. Guide member 114 comprises a proximal portion 530, a proximal spreader 640, a distal portion 532, a distal spreader 636, a guidewire exit lumen 626, and a guidewire tube 628. Guidewire tube 628 may be angled or curved to align fluidly with the guidewire lumen 437 of proximal shaft 108. FIG. 6 shows guide member 114 as it accesses guidewire lumen 437 of proximal shaft 108 without disturbing link lumen 438, link 332 or support shaft 343 of proximal shaft 108.

Guidewire exit port 123 is located in a recession 544 formed in proximal portion 530. Recession 544 aids insertion of guidewire 112 back through guidewire exit port 123, when feeding the guidewire 112 distally. Additionally, guidewire tube 628 extends distally from guidewire exit port 123 to intersect the guidewire lumen 437 in proximal shaft 108, preferably in a close coaxial relationship with the guidewire lumen 437. Once guidewire 112 is inserted into guidewire exit port 123, guidewire 112 passes through guidewire exit lumen 626, defined by guidewire tube 628, and into the guidewire lumen 437 of proximal shaft 108.

To ensure guidewire tube 628 accesses guidewire lumen 437 through longitudinal guide way 110, spreaders 636 and 640 open longitudinal guide way 110 as guide member 114 slides along proximal shaft 108. Once guide member 114 has passed, the resiliency of proximal shaft 108 closes longitudinal guide way 110. Spreaders 636 and 640 are formed in the body of guide member 114. Spreaders 636 and 640 also align guidewire tube 628 with longitudinal guide way 110. Once guidewire 112 has been led through the vasculature, guidewire 112 may be “backloaded” through guide member 114 by first being fed through guidewire exit port 111 of catheter tip 120, guidewire lumen 227, guidewire lumen 436, and guidewire lumen 437. As the guidewire 112 is fed into distal portion 532 of guide member 114, guidewire 112 will be fed into guidewire exit lumen 626 and out through guidewire exit port 123 rather than continuing proximally along guidewire lumen 437. Having the outer diameter of guidewire tube 628 just slightly smaller than the inner diameter of guidewire lumen 437 ensures that guidewire 112 will not likely be misfed through guide member 114.

Guide member 114 may be molded from a suitable rigid plastic material, for example, nylon, acrylonitrile-butadiene-styrene (ABS), a high density polyethylene, or PEBAX. Alternatively, guide member 114 may be made of a suitable metal, for example stainless steel, or guide member 114 may have both metal components and plastic components. For ease of manufacturing, guide member 114 may be comprised of molded parts that snap fit together. Guidewire tube 628 may be made from 304 stainless steel hypotube or a strong, thin-walled polymer, such as thermoset polyimide (PI) tubing or other comparable materials. Guidewire tube 628 may be fixed or slidably disposed in guide member 114.

To operate self-expanding stent delivery catheter 100, a clinician, for example, maneuvers guidewire 112 through a patient's vascular system until the distal end of guidewire 112 is positioned across the treatment site. In order for the clinician to maintain control over guidewire 112 during the loading of catheter 100, guide member 114 is slidably positioned along proximal shaft 108 at its distal most position along longitudinal guide way 110, just proximal of distal outer shaft 116. Catheter 100 is backloaded onto guidewire 112 until its proximal end exits guidewire exit port 123. Holding guidewire 112 and guide member 114 in a fixed position, proximal shaft 108 is then advanced distally over guidewire 112. As the shaft is advanced, guide member 114 slidingly opens the proximal portion of longitudinal guide way 110 of proximal shaft 108 and envelops more of guidewire 112 in guidewire lumen 437 of proximal shaft 108.

Catheter 100 is advanced over guidewire 112 until sheath 118 is positioned at the treatment site. Radiopaque markers 226 and 228 allow the clinician to monitor the location of the stent using a suitable technique, for example fluoroscopy. When the stent is properly positioned, the clinician slides sliding mechanism 106 in a proximal direction and in doing so mechanically slides distal outer shaft 116 in a proximal direction. As distal outer shaft 116 retracts, it slides relative to self-expanding stent 230 removing sheath portion 118. As sheath portion 118 continues to retract, the distal end of proximal radiopaque marker 226 keeps self-expanding stent 230 in place. As the distal end of distal outer shaft 116 is fully retracted, self-expanding stent 230 is free to deploy against a vessel wall. Once self-expanding stent 230 is in place, catheter 100 is then removed from the body and self-expanding stent 230 is left in place in the vessel.

Alternatively, guidewire 112 may be fully removed by pulling it out through guidewire exit port 123 while keeping catheter 100 in place. Once guidewire 112 is fully removed, a new guidewire may be introduced into guidewire exit port 123 and tracked through guidewire lumens 436, 437, 227 and guidewire exit port 111 to the treatment site.

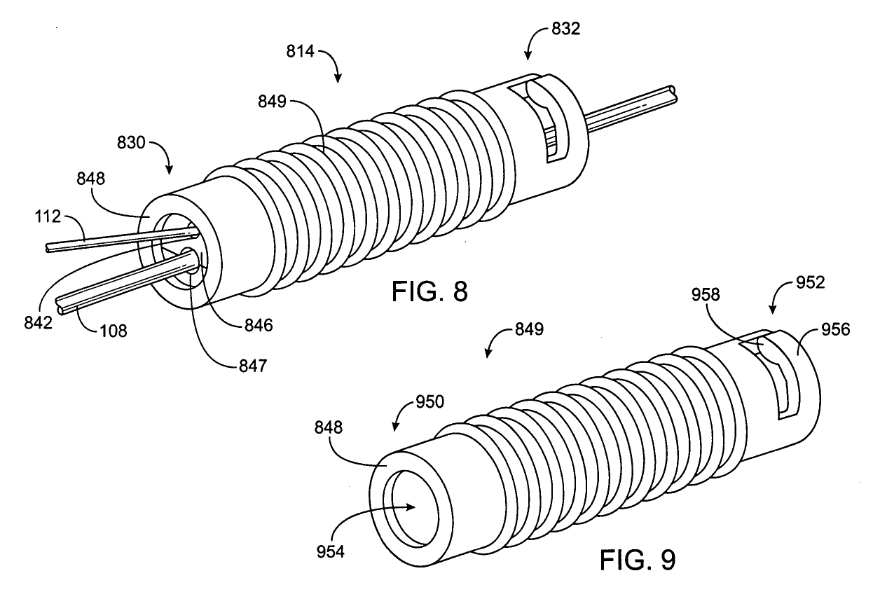

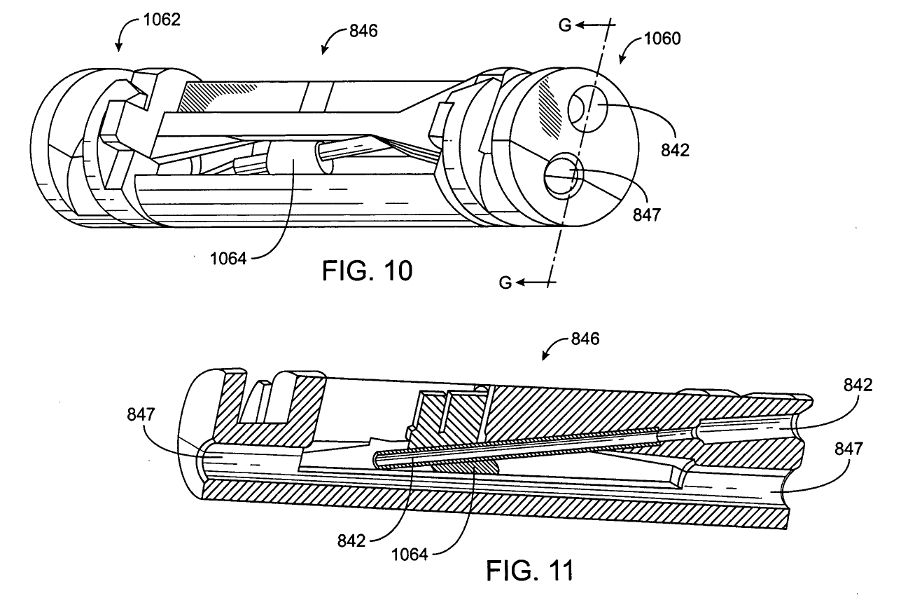

FIGS. 8-11 show an alternate embodiment of a guide member 814. In this instance, guide member 814 surrounds proximal shaft 108 and has a proximal end 830 and a distal end 832. Guide member 814 has an outer tubular member 849 with proximal and distal ends, 950 and 952 respectively, and a longitudinal bore 954 sized to receive an inner body 846. The outer tubular member 849 freely rotates about inner body 846 but is coupled to resist relative axial movement between outer tubular member 849 and inner body 846. A stop shoulder 848 positioned on proximal end 950 of the outer tubular member 849 consists of an annular wall radially extending into the longitudinal bore. The stop shoulder 848 prevents inner body 846 from slipping out of outer tubular member 849 through proximal end 950 of outer tubular member 849.

Two retaining arms 956 are disposed on distal end 952 of outer tubular member 849. Retaining arms 956 consist of two arcuate arms that form a portion of outer tubular member 849. Each arm 956 contains a tab 958 that extends into longitudinal bore 954 of outer tubular member 849 at its distal end 952. When guide member 814 is assembled, the tabs prevent inner body 846 from slipping out of the outer tubular member 849 through its distal end 952. Retaining arms 956 are flexible in the radial direction and may be flexed radially outward to temporarily remove tabs 958 from the longitudinal bore 954 to permit insertion and removal of inner body 846 during the assembly or disassembly of guide member 814. While two tabs 958 are shown positioned one hundred and eighty degrees apart, a different number of tabs may be used, provided they are spaced sufficiently to prevent inner body 846 from slipping out of the outer tubular member 849. Although the stop shoulder 848 and retaining arms 956 are described as integral parts of the outer tubular member, it should be understood that those features may be created by separate elements such as threaded caps.

Inner body 846, generally functions as guide member 114, of the previously discussed embodiment. Inner body 846 has proximal and distal ends, 1060 and 1062 respectively. Catheter receiving bore 847 extends longitudinally through inner body 846 from proximal end 1060 to distal end 1062. In the present embodiment, unlike the embodiment shown in FIG. 6, guide member 814 employs a single keel spreader member 1064. Keel spreader member 1064 serves to locally spread open longitudinal guide way 110 when guide member 814 is slidably mounted on proximal shaft 108. Guidewire passageway 842 extends through inner body 846 such that its distalmost end intersects catheter receiving bore 847 at a shallow angle, preferably ranging from about three to about fifteen degrees. Guidewire passageway 842 extends through keel spreader member 1064 to assure that guidewire 112 travels unobstructed through the spread longitudinal guide way 110, as shown in FIG. 11.

It shall be understood that the single keel 1064 design may be substituted for the dual spreader 536/540 design, shown in FIG. 6, and vice versa. In addition, like guide member 514, guide member 814 may be molded from a rigid plastic material, such as nylon or nylon based co-polymers, that is preferably lubricous. Alternatively, guide member 814 may be made of a suitable metal, such as stainless steel, or guide member 814 may have both metal components and plastic components. For ease in manufacturing, guide member 814 may be comprised of molded parts that snap-fit together to form the final configuration.

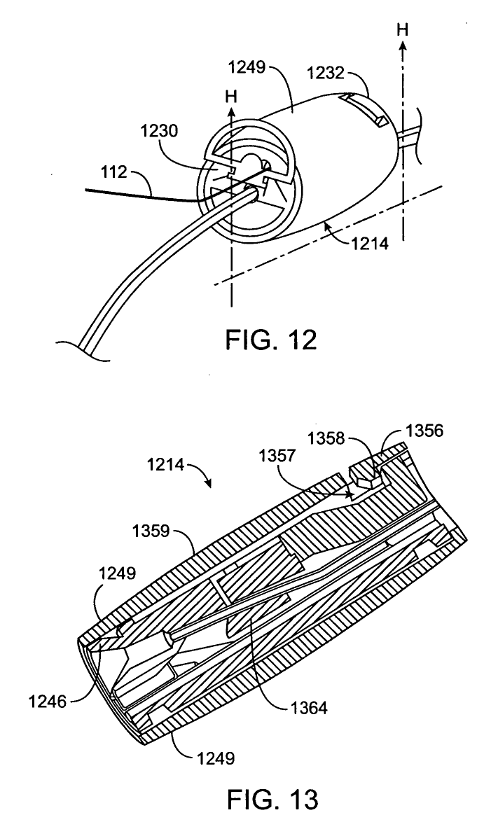

FIGS. 12-13 illustrate another embodiment of a guide member 1214. Guide member 1214 is similar to guide member 814, discussed above with respect to FIGS. 8-11. Guide member 814 surrounds proximal shaft 108 and has proximal end 1230 and distal end 1232. Guide member 1214 has an outer tubular member 1249 that freely rotates around an inner main body 1246 and a keel 1364, that functions identically to that of outer tubular member 849, inner main body 846, and keel 1064 of guide member 814. However, as shown in FIG. 9, retaining arm 1356 contains tab 1358 that extends into the space designated 1357 formed by main body 1246. Thus, when retaining arm 1356 is in the closed position, tab 1358 limits movement of main body 1246 since tab 1358 is captured within space 1357. Although only one tab 1358 is necessary, multiple tabs 1358 may be used. Further, while outer tubular member 849 of guide member 814 includes a textured surface with circumferential bosses to assist in grasping and manipulating guide member 814, outer tubular member 1249 has a smooth outer surface 1359.

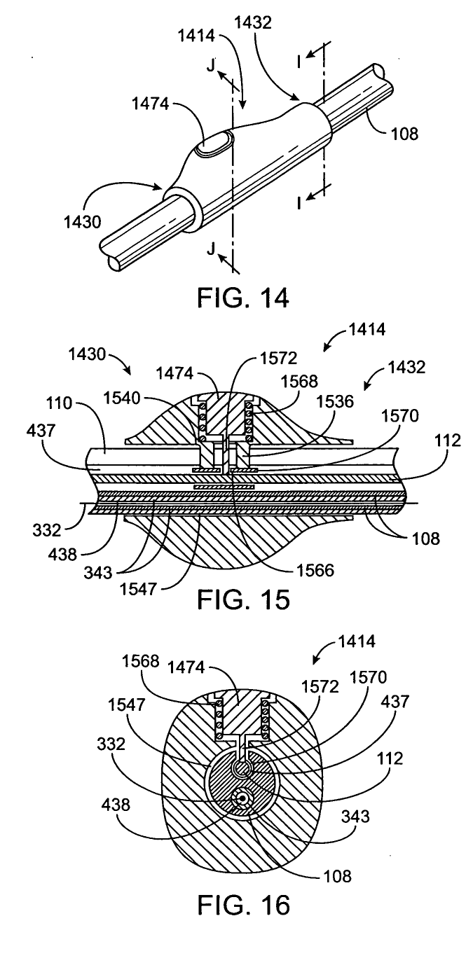

In FIGS. 14-16, a further alternative embodiment of a guide member 1414 is illustrated. In this embodiment, guide member 1414 is used to allow direct control over axial movement of guidewire 112. Such a guide member is disclosed in co-pending U.S. patent application Ser. No. 10/226,789, filed Aug. 21, 2002, the disclosure of which is incorporated by reference in its entirety herein. In FIG. 15, guidemember 1414 is shown accessing guidewire lumen 437 without disturbing link lumen 438, link 332, or support shaft 343 of proximal shaft 108.

Guide member 1414 has a main body having both proximal and distal ends, 1430 and 1432 respectively. A catheter receiving bore 1547 extends longitudinally through guide member 1414 from proximal end 1430 to distal end 1432. Guide member 1414 includes a proximal spreader member 1540 and a distal spreader member 1536 extending radially into catheter receiving bore 1547. In addition, a tubular guidewire receiver 1570 is mounted to proximal and distal spreader members, 1540 and 1536 respectively, within catheter receiving bore 1547 and is sized to slidably receive guidewire 112. The pair of spreader members serve to locally spread open longitudinal guide way 110 and provide a means for holding tubular guidewire receiver 1570 within guidewire lumen 437 when guide member 1414 is slidably mounted on proximal shaft 108. Tubular guidewire receiver 1570 has a side opening 1566 sized to receive a clamp member 1572. Proximal spreader member 1540 and distal spreader member 1536 serve to align proximal shaft 108 within catheter receiving bore 1547 and especially to align longitudinal guide way 110 with side opening 1566 on tubular guidewire receiver 1570.

Clamp member 1572 extends radially inward from a clamp control member 1474. Clamp control member 1474 and clamp member 1572 extend through the guide member 1414 and allow a clinician to manually engage a clamping force on the guidewire 112. In the present embodiment, a clamp spring 1568 is mounted to clamp control member 1474 and guide member 1414. Clamp spring 1568 holds clamp member 1572 and clamp control member 1474 in a disengaged state when no external force is placed on clamp control member 1474. When clamp control member 1474 is pressed and clamp spring 1568 is compressed, it causes clamp member 1572 to extend further radially into the catheter receiving bore 1547, through side opening 1566 in tubular guidewire receiver 1570 and against guidewire 112. That engagement with guidewire 112 results in a frictional force that resists relative movement between guidewire 112 and guide member 1414 allowing a practitioner to directly control the axial location of guidewire 112 within catheter 100.

Like guide members 114 and 814, guide member 1414 may be molded from a rigid plastic material, such as nylon or nylon based co-polymers, that is preferably lubricous. Alternatively, guide member 1414 may be made of a suitable metal, such as stainless steel, or guide member 1414 may have both metal components and plastic components. For ease in manufacturing, guide member 1414 may be comprised of molded parts that snap-fit together to form the final configuration.

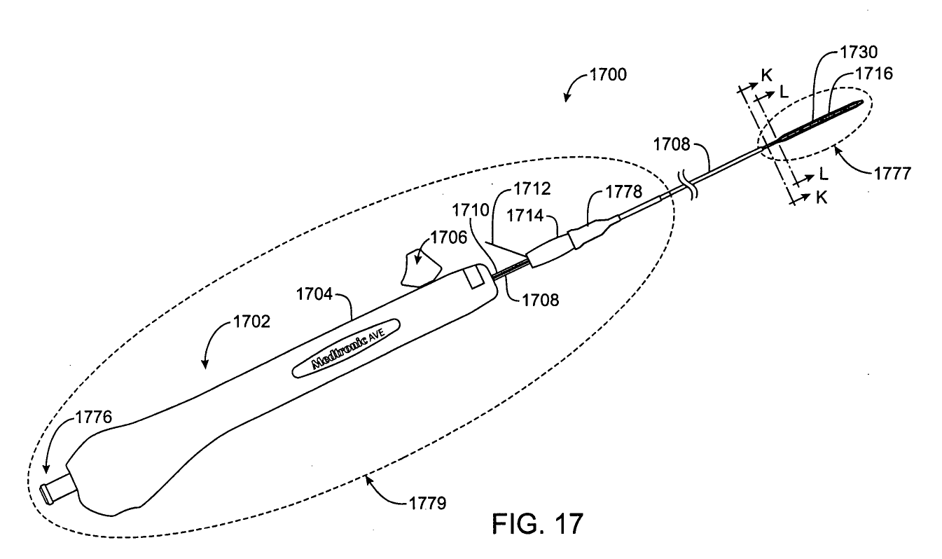

Another embodiment of the present invention catheter 1700 is illustrated in FIGS. 17, 18, 18A, 18B, and 19-23. As illustrated in FIG. 17, catheter 1700 includes a handle 1702 including a sliding mechanism 1706 and a luer 1776. Catheter 1700 also includes a proximal shaft 1708 extending from a distal end of handle 1702. A guide member 1714 and a relief valve 1778 are slidably connected to proximal shaft 108. A distal end of proximal shaft 108 is coupled to a proximal end of a distal outer shaft 1716, shown in partial cross-section, to include a self-expanding stent 1730, enclosed therein. Catheter 1770 operates and functions similarly to catheter 100 of FIG. 1. For example, guide member 1714 interacts with a longitudinal guide way 1710 running along the length of proximal shaft 1708 to access a guidewire 1712 and to withdraw guidewire 1712 from a guidewire lumen 1837 defined by proximal shaft 1708. However, instead of having a link 332 to retract distal outer shaft 116, catheter 1700 has distal outer shaft 1716 coupled to proximal shaft 1708, which is coupled to sliding mechanism 1706. As sliding mechanism 1706 is pulled proximally through a longitudinal opening 1704 in handle 1702, proximal shaft 1708 retracts along with distal outer shaft 1716 to release stent 1730.

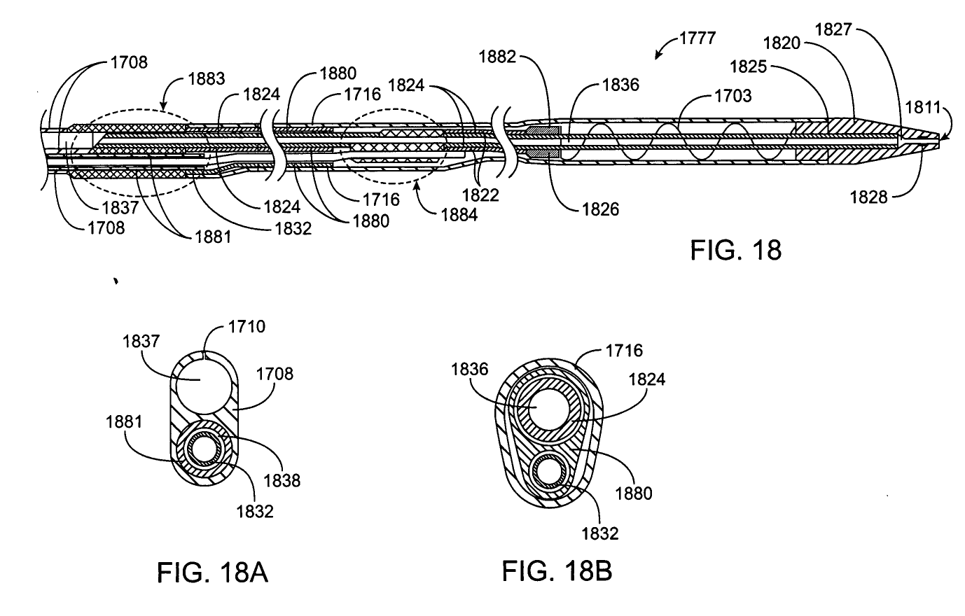

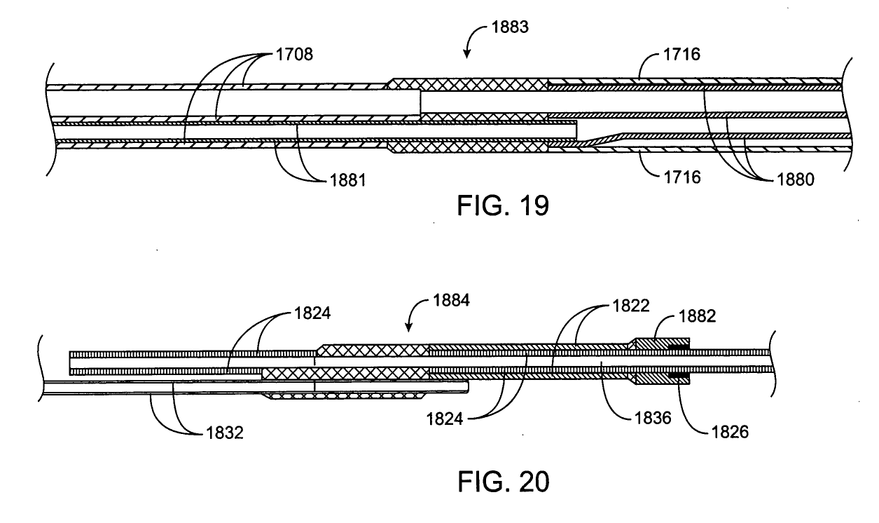

FIGS. 18, 18A and 18B illustrates an expanded view of the distal end 1777 of catheter 1700 of FIG. 17, with FIGS. 18A and 18B being cross-sectional views taken along lines K-K and L-L, respectively, of FIG. 17. FIG. 18 shows where proximal shaft 1708 is bonded to distal outer shaft 1716 and an intermediate shaft 1880. The bonding region 1883 between proximal shaft 1708, distal outer shaft 1716 and intermediate shaft 1880 is shown in an expanded view in FIG. 19. In another embodiment, intermediate shaft 1880 may be, for example, merely a continuation of proximal shaft 1708. However, having a separate intermediate shaft 1880 allows for a shaft made from a different material, for example a stiffer material, or for a shape of a shaft that is different from proximal shaft 1708, as shown in FIGS. 18A and 18B. Proximal shaft 1708 defines a guidewire lumen 1837.

Proximal shaft 1708 also includes a support shaft 1881 bonded thereto, which defines a link lumen 1838. Link lumen 1838 has a link 1832. Link 1832 may be, for example, a pushtube, such as the flush hypotubing shown in FIG. 18, or a pushwire. A pushtube has the added advantage of being able to administer fluids, such as saline, dyes or drugs, to a treatment site from luer 1776 proximal of handle 1702.

As shown in detail in FIG. 20, which is shown without distal outer shaft 1716, link 1832 is bonded to a distal inner shaft 1824 at a bonding site 1884. Link 1832 is used to hold distal inner shaft 1824 in a distal position as proximal shaft 1708 and distal outer shaft 1716 are retracted. Distal inner shaft 1824 also defines a guidewire lumen 1836 which communicates with guidewire lumen 1837 of proximal shaft 1708.

Coupled to an exterior of distal inner shaft 1824 is a spacer shaft 1822. Spacer shaft 1822 has a distal end including a stop flange 1882. Stop flange 1882 includes a proximal radiopaque marker 1826. Stop flange 1882 has an outer diameter that is slightly smaller than the inner diameter of distal outer shaft 1716. Stent 1730 is positioned between an exterior of distal inner shaft 1824 and an interior surface of distal outer shaft 1716, and distally of stop flange 1882 of spacer shaft 1822. As such, stop flange 1882 prevents stent 1730 from sliding proximally as catheter 1700 is advanced through the vasculature and as distal outer shaft 1716 is retracted proximally for deployment of stent 1730. The interior surface of distal outer shaft 1716 may include a lubricious material, such as silicone or a hydrophilic material, to allow the coated inner surface to easily slide relative to stent 1730.

A distal end of distal inner shaft 1824 is coupled to a catheter tip 1820. Catheter tip 1820 defines a guidewire lumen 1827 and a guidewire exit port 1811. Catheter tip 1820 also includes a distal radiopaque marker 1828, which may be positioned as shown in FIG. 18 to identify the distal end of catheter 1700 or it may be positioned in a proximal end of catheter tip 1820 to identify the distal end of stent 1730. Catheter tip 1820 includes a groove 1825 which receives a distal end of distal outer shaft 1716, when in a distal position.

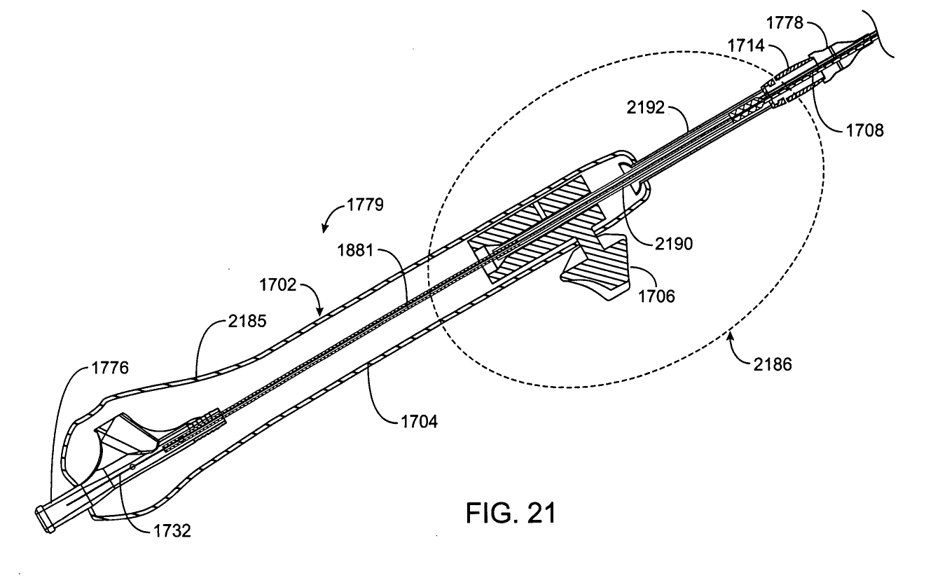

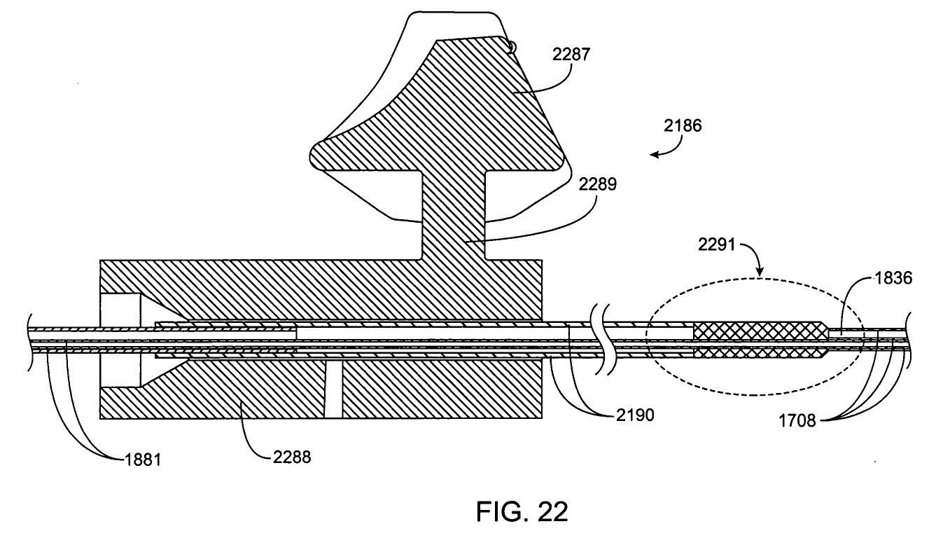

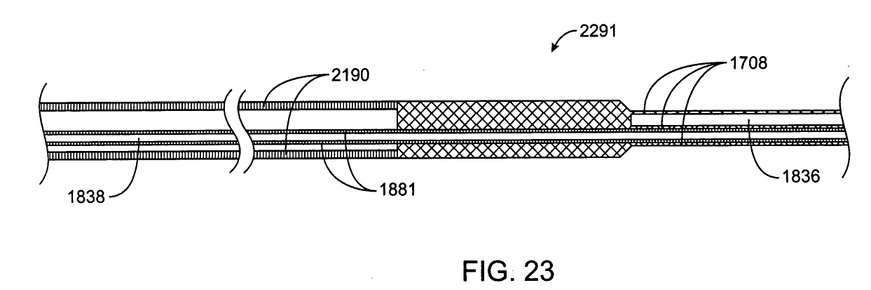

FIG. 21 illustrates an expanded cross-sectional view of the proximal end 1779 of catheter 1700 of FIG. 17. Handle 1702 includes a casing 2185 with a longitudinal opening 1704. A proximal end of handle 1702 includes a luer 1776 through which link 1732 may be accessed. Link 1732 runs the length of the handle 1702, while support shaft 1881 defining link lumen 1838, which moved in conjunction with proximal shaft 1708, stops shy of the proximal end of handle 1702 so that it will have room to move within handle 1702. Handle 1702 further includes sliding mechanism 1706. A detailed view of the sliding mechanism connection 2186 of handle 1702 is shown in FIGS. 22 and 23. As illustrated in FIG. 22, sliding mechanism includes a knob 2287 coupled to a tubular region 2288 by post 2289. Tubular region 2288 is bonded to a primary shaft 2190. Primary shaft 2190 is bonded to proximal shaft 1708 at a bonding region 2291, which is shown in detail in FIG. 23. Primary shaft 2190 does not extend distally beyond handle 1702, and as such may have a larger diameter than proximal shaft 1708, which must be smaller as it is directed through the vasculature of a patient. In an alternate embodiment, sliding mechanism 1706 may be bonded directly to proximal shaft 1708.

As shown in FIG. 21, a distal end of handle 1702 may include an optional exterior shaft 2192, which prevents guide member 1714 and valve relief 1778 from moving proximally as proximal shaft 1708 is retracted. Guide member 1714 is not shown in detail in FIG. 21, because it may be any of the guide members described or otherwise disclosed herein above.

Guide member 1714 is bonded to valve relief 1778. The manually adjustable gasket of a “Tuohy-Borst” fitting, when tightened around a proximal shaft 1708 at the entrance to the body of a patient, does not allow the proximal shaft 1708 to move relative to the gasket. As such, any time the proximal shaft 1708 needs to move, the gasket must be loosened and retightened. Each time the gasket is loosened, backbleeding occurs through the incision. In this embodiment, the “Tuohy-Borst” gasket is connected to valve relief 1778. Both guide member 1714 and valve relief 1778 then maintain a fixed position just exterior to the body of the patient. Thus, rather than moving proximal shaft 1708 through the gasket, proximal shaft 1708 moves in and out of the body through valve relief 1778 without affecting the gasket seal and without as much backbleeding. Proximal shaft 1708 may also rotate within valve relief 1778 so that proximal shaft 1708 may be twisted and turned through tortuous vasculature from a distal position.

While this invention has been particularly shown and described with reference to preferred embodiments thereof, it will be understood by those skilled in the art that various changes in form and details may be made therein without departing from the spirit and scope of the invention.

What is claimed is:

1. A catheter for a self-expanding stent, comprising:
a guidewire lumen defined by a proximal shaft, said proximal shaft including a longitudinal guide way extending substantially from a proximal end to a distal end of said proximal shaft and radially from an exterior surface of said proximal shaft to said guidewire lumen;
a guide member slidably attached to said proximal shaft and including a guidewire exit lumen accessing said guidewire lumen via said longitudinal guide way of said proximal shaft;
a sliding mechanism coupled to said proximal end of said proximal shaft; and
a distal outer shaft coupled to said sliding mechanism, wherein said distal outer shaft retains a self-expanding stent when in a distal position and releases said self-expanding stent when in a proximal position.

2. The catheter of claim 1, wherein said distal outer shaft is coupled to said sliding mechanism via said proximal shaft.

3. The catheter of claim 1, wherein said distal outer shaft is coupled to said sliding mechanism via a link disposed within a link lumen of said proximal shaft.

4. The catheter of claim 3, wherein said link is a pullwire or a pulltube.

5. The catheter of claim 1, wherein said sliding mechanism is disposed within a handle.

6. The catheter of claim 1, further comprising: a distal inner shaft, said distal inner shaft having a distal end coupled to a catheter tip, wherein said distal inner shaft and said catheter tip define a guidewire lumen.

7. The catheter of claim 6, wherein a proximal end of said distal inner shaft is coupled to said proximal shaft.

8. The catheter of claim 6, wherein said distal inner shaft is coupled to a link slidably positioned within said proximal shaft.

9. The catheter of claim 6, wherein said distal inner shaft is coupled to a spacer shaft, said spacer shaft including a stop capable of stopping said self-expanding stent from moving proximally and a radiopaque marker.

10. The catheter of claim 6, wherein said catheter tip includes a radiopaque marker.

11. A self-expanding stent delivery catheter comprising:
a handle;
a proximal shaft defining a first guidewire lumen and having a proximal end coupled to said handle and a closeable longitudinal guide way between an exterior surface of said proximal shaft and said first guidewire lumen;
a guide member slidably attached to said proximal shaft, wherein said guide member includes a guidewire exit lumen accessing the first guidewire lumen via said closable longitudinal guide way;
a distal inner shaft having a proximal end connected to a distal end of said proximal shaft and having a second guidewire lumen with a proximal end in communication with a distal end of said first guidewire lumen; and
a distal outer shaft longitudinally slidable relative to a portion of each of said proximal shaft and said distal inner shaft;
a sliding mechanism disposed within said handle and coupled to said distal outer shaft via a link; and
a self-expanding stent positioned between an exterior surface of said distal inner shaft and an interior surface of said distal outer shaft.

12. The catheter of claim 11, wherein said link is a pullwire or a pulltube.

13. The catheter of claim 11, wherein said handle further includes a longitudinal groove in which said sliding mechanism slides proximally and distally.

14. The catheter of claim 11, further comprising: a spacer shaft positioned along the exterior surface of said distal inner shaft, said spacer shaft being connected at a proximal end to a distal end of said proximal shaft and at a distal end to a radiopaque marker.

15. The catheter of claim 11, wherein said distal outer shaft further includes a proximal portion and a bump distal portion, wherein said proximal portion has a smaller diameter than said bump distal portion.

16. The catheter of claim 11, further comprising: a catheter tip connected to a distal end of said distal inner shaft, wherein the catheter tip includes a third guidewire lumen and a distal guidewire exit port such that a proximal end of said third guidewire lumen is in communication with a distal end of said second guidewire lumen.

17. The catheter of claim 16, wherein said self-expanding stent is positioned distal to a radiopaque marker and proximal to said catheter tip.

18. The catheter of claim 17, wherein said self-expanding stent is longitudinally positioned proximal to a second radiopaque marker located in said catheter tip.

19. The catheter of claim 16, wherein said catheter tip includes a groove which engages a distal end of said distal outer shaft.

20. A self-expanding stent delivery catheter comprising:
a proximal shaft having a proximal end and a distal end;
a distal inner shaft having a proximal end and a distal end, said proximal end being connected to said distal end of said proximal shaft;
a distal outer shaft positioned exterior to and slidable with respect to a portion of said proximal shaft and said distal inner shaft;
wherein said proximal shaft and said distal inner shaft define a guidewire lumen; said proximal shaft including a longitudinal guide way between an exterior surface and said guidewire lumen;
a guide member slidably attached to said exterior surface of said proximal shaft and defining a guidewire exit lumen for accessing said guidewire lumen via said longitudinal guide way; and
a self expanding stent positioned between said distal outer shaft and said distal inner shaft.

21. The catheter of claim 20, further comprising: a sliding mechanism positioned proximal to said proximal end of said proximal shaft and coupled to said distal outer shaft via a link, wherein said sliding mechanism is capable of pulling said distal outer shaft proximally via said link to release said self-expanding stent

22. The catheter of claim 20, wherein said self-expanding stent is located longitudinally between a proximal radiopaque marker and a distal radiopaque marker.

23. The catheter of claim 20, further comprising: a stop positioned proximal to said self-expanding stent, said stop being capable of stopping said self-expanding stent from sliding proximally.

24. The catheter of claim 23, wherein said stop is one of a radiopaque marker or a stop flange.

25. The catheter of claim 21, wherein said link is one of a pullwire or a pulltube.

26. A catheter for delivering a self-expandable stent comprising:
a guidewire lumen defined by a proximal shaft and a distal inner shaft, said proximal shaft including a longitudinal guide way extending from a proximal end to a distal end of said proximal shaft and radially from an exterior surface of said proximal shaft to said guidewire lumen;
a sliding mechanism coupled to said proximal end of said proximal shaft;
a distal outer shaft coupled to said distal end of said proximal shaft and positioned exteriorly to said distal inner shaft, such that said proximal shaft and said distal shaft are capable of sliding with respect to said distal inner shaft;
a guide member slidably attached to said proximal shaft and including a guidewire exit lumen for accessing said guidewire lumen via said longitudinal guide way of said proximal shaft; and
a self-expanding stent positioned between an exterior of said distal inner shaft and an interior of said distal outer shaft, wherein said sliding mechanism is capable of pulling said proximal shaft and said distal shaft proximally to release said self-expanding stent.

27. The catheter of claim 26, wherein said sliding mechanism is disposed within a handle.

28. The catheter of claim 26, further comprising: a valve relief coupled to said guide member and slidably positioned around an exterior of said proximal shaft.

29. The catheter of claim 26, further comprising: a link, said link having a proximal end accessible via a luer and a distal end coupled to said distal inner shaft.

30. The catheter of claim 29, wherein said link is one of a pullwire or a pulltube.

31. The catheter of claim 29, wherein said link is a flush hypotube.

32. The catheter of claim 29, wherein said link is slidably positioned within a link lumen defined by a support shaft, said support shaft being coupled to said proximal shaft.

33. The catheter of claim 26, further comprising:
a spacer shaft coupled to an exterior of said distal inner shaft, said spacer shaft having a distal end positioned proximal of said self-expanding stent and including a stop flange capable of stopping said self-expanding stent from moving proximally.

34. The catheter of claim 33, wherein said stop flange includes a radiopaque marker.

35. The catheter of claim 26, wherein said distal inner shaft is coupled to a catheter tip further defining said guidewire lumen and including a guidewire exit port.

36. The catheter of claim 35, wherein said catheter tip further includes a radiopaque marker.

37. The catheter of claim 35, wherein said catheter tip includes a groove for slidably receiving a distal end of said distal outer shaft.
